(19) United States
(12) Patent Application Publication
(10) Pub. No.: US 2014/0364739 A1
(43) Pub. Date: Dec. 11, 2014

(54) SYSTEMS AND METHODS FOR ANALYZING A VASCULAR STRUCTURE

(71) Applicant: General Electric Company, Schenectady, NY (US)

(72) Inventors: Shubao Liu, Niskayuna, NY (US); Paulo Ricardo Mendonca, Niskayuna, NY (US); Dirk Ryan Padfield, Niskayuna, NY (US)

(73) Assignee: General Electric Company, Schenectady, NY (US)

(21) Appl. No.: 13/911,869

(22) Filed: Jun. 6, 2013

(51) Int. Cl. A61B 8/08; A61B 6/03; A61B 8/13; A61B 5/02; A61B 5/107

(52) U.S. Cl. CPC A61B 8/5223; A61B 5/02007; A61B 5/1075; A61B 8/0891; A61B 8/13; A61B 6/032; A61B 6/037
USPC 600/449; 600/407

(57) ABSTRACT

System for analyzing a vascular structure. The system includes an initialization module that is configured to analyze a slice of a VOI that includes a main vessel of the vascular structure to position first and second luminal models in the lumen. Each of the first and second luminal models represents at least a portion of a cross-sectional shape of the lumen and has a location and a dimension in the slice. The system also includes a tracking module that is configured to determine the locations and the dimensions of the first and second luminal models in subsequent slices. For a designated slice, the locations and the dimensions of the first and second luminal models of the designated slice are based on the locations and the dimensions of the first and second luminal models, respectively, in a prior slice and also the image data of the designated slice.

### BACKGROUND

The subject matter disclosed herein relates generally to modeling and/or analyzing vascular structures using medical image data, and more particularly to vascular structures that include at least one bifurcation or branching of vessels.

Cardiovascular disease (CVD) is the leading cause of death worldwide. Like other diseases, early diagnosis of CVD may increase the number and effectiveness of treatments and consequently increase survival and/or quality of life of a patient. The presence of plaque in the cardiovascular system has been identified as a risk factor in the prognosis of CVD. Medical imaging modalities that have been used to detect the presence of plaque in a vascular structure include magnetic resonance (MR), computed tomography (CT), and ultrasound.

Challenges may exist, however, for imaging particular vascular structures. For instance, it may be difficult to reliably image (or model through imaging) a vascular structure that includes one or more regions in which a vessel branches into multiple vessels. By way of one specific example, the carotid includes a main vessel, which is referred to as the common carotid artery (CCA). The CCA has an enlarged portion, referred to as a bulb region, which includes a bifurcation in which the carotid separates into two branches. The two branches are referred to as the external carotid artery (ECA) and the internal carotid artery (ICA). Blood initially flows through the CCA and is then divided into the ECA and ICA. Imaging vascular structures that include bifurcations, such as the carotid, may be challenging due to noise, adjacent vascular structures, anatomical variations in the patient population, and motion caused by patient respiration and/or cardiac pulsation. When ultrasound imaging is used, another challenge may be to account for unstable movement of the ultrasound probe and images that have a lower quality compared to other imaging modalities.

The presence of plaque in the carotid has been identified as a significant risk factor in the prognosis of CVD. Accordingly, reliable systems for locating the carotid walls and/or analyzing carotid plaque are desired. It may be particularly desirable to have systems that are capable of locating carotid walls and/or analyzing carotid plaque with minimal interaction by a technician. Although some methods and systems have been proposed for analyzing medical images and identifying vessel walls and/or plaque markers, these methods and systems may require significant user input, extensive technical training for effective image acquisition, and/or high-quality images (e.g., CT or MR images). Commonly used segmentation algorithms (e.g., level-set segmentation, snakes, region growing, and the like) may not be effective for analyzing ultrasound images due to the low image quality and local ambiguities in the images.

### BRIEF DESCRIPTION

In one embodiment, a system for analyzing a vascular structure is provided. The vascular structure has a main vessel and first and second vessels that branch from the main vessel. Each of the main vessel and the first and second vessels has a respective lumen. The system includes an initialization module that is configured to receive image data of a volume-of-interest (VOI) that includes the vascular structure, wherein the VOI is represented as a series of slices that are taken substantially transverse to a flow of blood through the vascular structure. The initialization module is configured to analyze a slice of the VOI that includes the main vessel to position first and second luminal models in the lumen of the main vessel. Each of the first and second luminal models represents at least a portion of a cross-sectional shape of the respective lumen and has a location and a dimension in the slice. The system also includes a tracking module that is configured to determine the locations and the dimensions of the first and second luminal models in subsequent slices. The first and second luminal models follow along paths of the first and second vessels, respectively. For a designated slice, the locations and the dimensions of the first and second luminal models of the designated slice are based on the locations and the dimensions of the first and second luminal models, respectively, in a prior slice and also the image data of the designated slice.

In another embodiment, a method for analyzing a vascular structure is provided. The method includes receiving image data of a volume-of-interest (VOI) that has the vascular structure. The vascular structure includes a main vessel and first and second vessels that branch from the main vessel. Each of the main vessel and the first and second vessels having a respective lumen, wherein the VOI is represented as a series of slices that are taken substantially transverse to a flow of blood through the vascular structure. The method includes analyzing a slice of the VOI that includes the main vessel to position first and second luminal models in the lumen of the main vessel. Each of the first and second luminal models represents at least a corresponding portion of a cross-sectional shape of the respective lumen and has a location in the slice and a dimension. The method also includes determining the locations and the dimensions of the first and second luminal models in subsequent slices. The first and second luminal models follow paths of the first and second vessels, respectively. For a designated slice, the locations and the dimensions of the first and second luminal models of the designated slice are based on the locations and the dimensions of the first and second luminal models, respectively, in a prior slice and also the image data of the designated slice. The method also includes using the locations and the dimensions of the first and second luminal models to image or analyze the vascular structure.

In another embodiment, an ultrasound system is provided that includes an ultrasound probe configured to acquire ultrasound image data of a vascular structure. The vascular structure has a main vessel and first and second vessels that branch from the main vessel. Each of the main vessel and the first and second vessels has a respective lumen. The system includes an initialization module that is configured to receive image data of a volume-of-interest (VOI) that includes the vascular structure, wherein the VOI is represented as a series of slices that are taken substantially transverse to a flow of blood through the vascular structure. The initialization module is configured to analyze a slice of the VOI that includes the main vessel to position first and second luminal models in the lumen of the main vessel. Each of the first and second luminal models represents at least a portion of a cross-sectional shape of the respective lumen and has a location and a dimension in the slice. The system also includes a tracking module that is configured to determine the locations and the dimensions of the first and second luminal models in subsequent slices. The first and second luminal models follow along paths of the first and second vessels, respectively. For a designated slice, the locations and the dimensions of the first and second luminal models of the designated slice are based on the locations and the dimensions of the first and second luminal models, respectively, in a prior slice and also the ultrasound data of the designated slice.

### BRIEF DESCRIPTION OF THE DRAWINGS

FIG. 1 illustrates a block diagram of a system for analyzing a vascular structure in accordance with one embodiment.

FIG. 2 is a flowchart illustrating a method of analyzing a vascular structure in accordance with one embodiment.

FIG. 3 illustrates a vascular structure and includes representative slices of the vascular structure.

FIG. 4 illustrates the process of locating luminal models in a subsequent slice based on the locations and dimensions of luminal models in a prior slice.

FIG. 5 illustrates an average image of a single vessel that is based on a plurality of images of different vessels.

FIG. 6 is a graph illustrating an average radial profile of a single vessel.

FIG. 7 illustrates slices of four different carotids that were analyzed by an embodiment described herein.

FIG. 8 illustrates respective surface renderings of the four different carotids shown in FIG. 7.

### DETAILED DESCRIPTION

Embodiments described herein include systems, methods, and computer readable media that may be used to image, analyze, and/or model vascular structures within a volume of interest (VOI) of a patient (human or animal) using image data. At least some embodiments may be used to facilitate characterizing and/or measuring plaque that is located along an interior surface of a vessel wall. The vascular structure may include at least one branch region (e.g., bifurcation) in which a main vessel (also referred to as a common vessel) branches into two or more vessels or, alternatively, in which two or more vessels converge into a main vessel. The image data may be obtained through one or more medical imaging modalities that are suitable for imaging vascular systems, such as magnetic resonance (MR), computed tomography (CT), and ultrasound. Other non-limiting examples of medical imaging modalities may include positron emission tomography (PET) and single photon emission computed tomography (SPECT). Embodiments may be particularly useful for cases in which the image data is of a relatively lower quality, such as in ultrasound. In some embodiments, the methods set forth herein may be entirely automated after the image data is obtained. In other embodiments, some user intervention may be permitted or used (e.g., one or more inputs may be entered by the user).

A vascular model (or rendering or image) of the vascular structure may be generated by analyzing portions or subsets of image data, which are hereinafter referred to as slices (or frames), and determining one or more luminal models for each slice. Each luminal model may represent a location, shape (or contour), and size or dimension of a lumen of one of the vessels (e.g., main or branch vessels). As described herein, the luminal model in a slice-of-interest may be based on, at least in part, a previously calculated luminal model from a prior slice and also image data that corresponds to the slice-of-interest. The luminal models in a slice-of-interest may also be based on an appearance model or template, which may represent an expected image or optimal image of the anatomy at the slice-of-interest.

In particular embodiments, the luminal models may be calculated using a model-selection function. The model-selection function may determine which parameter, from a number of potential parameters, provides the more suitable luminal model for the slice. In some embodiments, the model-selection function may compare the acquired image data that corresponds to the slice to an appearance model or template, such as those described herein. The model-selection function may determine the more suitable luminal model for a designated slice from a limited number of potential models. The model-selection function may be similar to a likelihood function that determines the parameter(s) that has the greatest likelihood or a cost function that determines the parameter(s) that has the least cost. The model-selection function may be a probability distribution function. Notably, embodiments described herein may automatically identify one or more luminal models for an initial slice and then automatically identify the luminal models for the subsequent slices for the entire vascular structure. After determining the luminal models for each slice, the luminal models may be collectively used to generate an image (e.g., surface rendering) and/or analyze the vascular structure in the VOI.

The following detailed description of various embodiments will be better understood when read in conjunction with the appended figures. To the extent that the figures illustrate diagrams of the functional blocks of the various embodiments, the functional blocks are not necessarily indicative of the division between hardware circuitry. Thus, for example, one or more of the functional blocks (e.g., modules, processing units, or memories) may be implemented in a single piece of hardware (e.g., a general purpose signal processor or a block of random access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

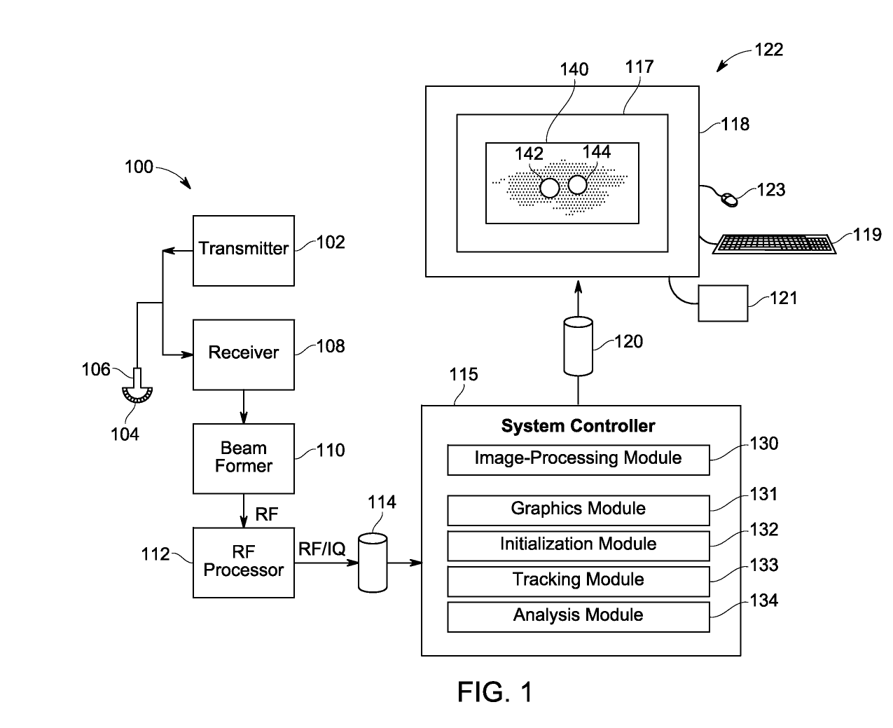

FIG. 1 illustrates a block diagram of a system 100 according to one embodiment. In the illustrated embodiment, the system 100 is an imaging system and, more specifically, an ultrasound imaging system. However, it is understood that embodiments set forth herein may be implemented using other types of medical imaging modalities (e.g., MR, CT, PET/CT, etc.). Furthermore, it is understood that other embodiments do not actively acquire medical images. Instead, embodiments may retrieve image data that was previously acquired by an imaging system and analyze the image data as set forth herein. As shown, the system 100 includes multiple components. The components may be coupled to one another to form a single structure, may be separate but located within a common room, or may be remotely located with respect to one another. For example, one or more of the modules described herein may operate in a data server that has a distinct and remote location with respect to other components of the system 100, such as a probe and user interface. Optionally, in the case of ultrasound systems, the system 100 may be a unitary system that is capable of being moved (e.g., portably) from room to room. For example, the system 100 may include wheels or be transported on a cart.

In the illustrated embodiment, the system 100 includes a transmitter 102 that drives an array of elements 104, for example, piezoelectric crystals, within a diagnostic ultrasound probe 106 (or transducer) to emit pulsed ultrasonic signals into a body or volume (not shown) of a subject. The elements 104 and the probe 106 may have a variety of geometries. The ultrasonic signals are back-scattered from structures in the body, for example, blood vessels and surrounding tissue, to produce echoes that return to the elements 104. The echoes are received by a receiver 108. The received echoes are provided to a beamformer 110 that performs beamforming and outputs an RF signal. The RF signal is then provided to an RF processor 112 that processes the RF signal. Alternatively, the RF processor 112 may include a complex demodulator (not shown) that demodulates the RF signal to form IQ data pairs representative of the echo signals. The RF or IQ signal data may then be provided directly to a memory 114 for storage (for example, temporary storage).

The system 100 also includes a system controller 115 that includes a plurality of modules, which may be part of a single processing unit (e.g., processor) or distributed across multiple processing units. The system controller 115 is configured to control operation of the system 100. For example, the system controller 115 may include an image-processing module 130 that receives image data (e.g., ultrasound signals in the form of RF signal data or IQ data pairs) and processes image data. For example, the image-processing module 130 may process the ultrasound signals to generate slices or frames of ultrasound information (e.g., ultrasound images) for displaying to the operator. When the system 100 is an ultrasound system, the image-processing module 130 may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. The generated ultrasound images may be two-dimensional (2D) or three-dimensional (3D). When multiple two-dimensional (2D) images are obtained, the image-processing module 130 may also be configured to stabilize or register the images through, for example, inter-slice registration as described below.

Acquired ultrasound information may be processed in real-time during an imaging session (or scanning session) as the echo signals are received. Additionally or alternatively, the ultrasound information may be stored temporarily in the memory 114 during an imaging session and processed in less than real-time in a live or off-line operation. An image memory 120 is included for storing processed slices of acquired ultrasound information that are not scheduled to be displayed immediately. The image memory 120 may comprise any known data storage medium, for example, a permanent storage medium, removable storage medium, and the like.

In operation, an ultrasound system may acquire data, for example, volumetric data sets by various techniques (for example, 3D scanning, real-time 3D imaging, volume scanning, 2D scanning with transducers having positioning sensors, freehand scanning using a voxel correlation technique, scanning using 2D or matrix array transducers, and the like). Ultrasound images 125 of the system 100 may be displayed to the operator or user on the display device 118.

The system controller 115 is operably connected to a user interface 122 that enables an operator to control at least some of the operations of the system 100. The user interface 122 may include hardware, firmware, software, or a combination thereof that enables an individual (e.g., an operator) to directly or indirectly control operation of the system 100 and the various components thereof. As shown, the user interface 122 includes a display device 118 having a display area 117. In some embodiments, the user interface 122 may also include one or more input devices, such as a physical keyboard 119, mouse 123, and/or touchpad 121. In an exemplary embodiment, the display device 118 is a touch-sensitive display (e.g., touchscreen) that can detect a presence of a touch from the operator on the display area 117 and can also identify a location of the touch in the display area 117. The touch may be applied by, for example, at least one of an individual's hand, glove, stylus, or the like. As such, the touch-sensitive display may also be characterized as an input device that is configured to receive inputs from the operator. The display device 118 also communicates information to the operator by displaying the information to the operator. The display device 118 and/or the user interface 122 may also communicative audibly. The display device 118 is configured to present information to the operator during the imaging session. The information presented may include ultrasound images, graphical elements, user-selectable elements, and other information (e.g., administrative information, personal information of the patient, and the like).

In addition to the image-processing module 130, the system controller 115 may also include a graphics module 131, an initialization module 132, a tracking module 133, and an analysis module 134. The image-processing module 130, the graphics module 131, the initialization module 132, the tracking module 133, and the analysis module 134 may coordinate with one another to present information to the operator during and/or after the imaging session. For example, the image-processing module 130 may be configured to display an acquired image 140 on the display device 118, and the graphics module 131 may be configured to display designated graphics along with the ultrasound image, such as graphical outlines 142, 144, which represent lumens or vessel walls in the acquired image 140. The image-processing and/or graphics modules 130, 131, may also be configured to generate a 3D rendering or image (not shown) of the entire vascular structure.

It is noted that although one or more embodiments may be described in connection with an ultrasound imaging system, the embodiments described herein are not limited to ultrasound imaging systems. In particular, one or more embodiments may be implemented in connection with different types of medical imaging systems. Examples of such medical imaging systems include a magnetic resonance imaging (MRI) system, computed tomography (CT) system, positron emission tomography (PET) system, a PET/CT system, and single photon emission computed tomography (SPECT) system. In such embodiments, the acquired images may be MRI images, CT images, PET images, PET/CT images, and SPECT images.

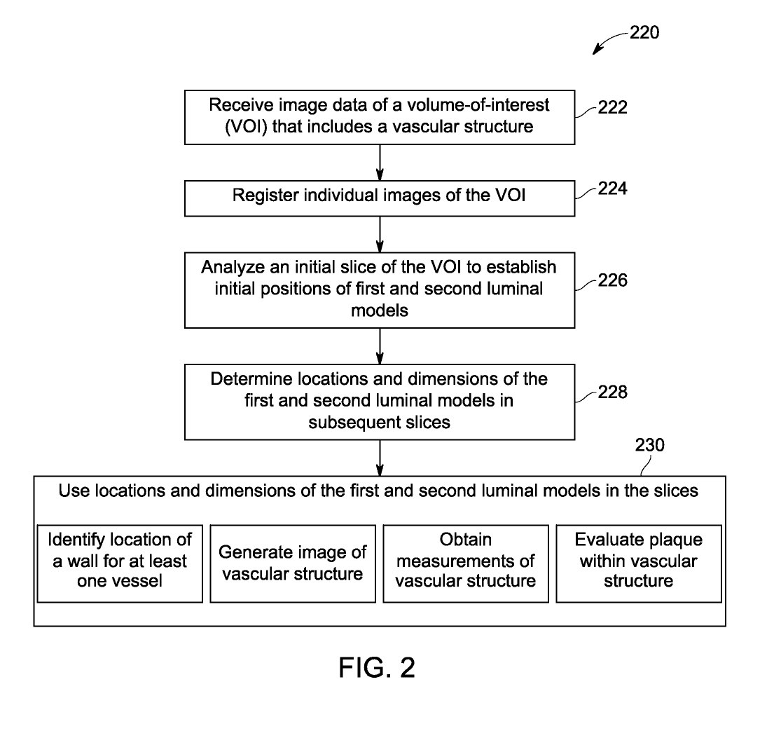

FIG. 2 is a flowchart illustrating a method 220 for analyzing a vascular structure, such as the vascular structure 200 (FIG. 2). The method 220 may employ structures or aspects of various embodiments described herein, such as the system 100. In particular, the operations shown in FIG. 2 may be executed by one or more of the modules 130-134. In various embodiments, certain operations (or steps) may be omitted or added, certain operations may be combined, certain operations may be performed simultaneously, certain operations may be performed concurrently, certain operations may be split into multiple sub-operations, certain operations may be performed in a different order, or certain operations or series of operations may be re-performed in an iterative fashion. Each of the operations of the method 220 may be performed automatically by the system 100. In other embodiments, the system 100 may prompt the user for inputs to proceed with executing the operations.

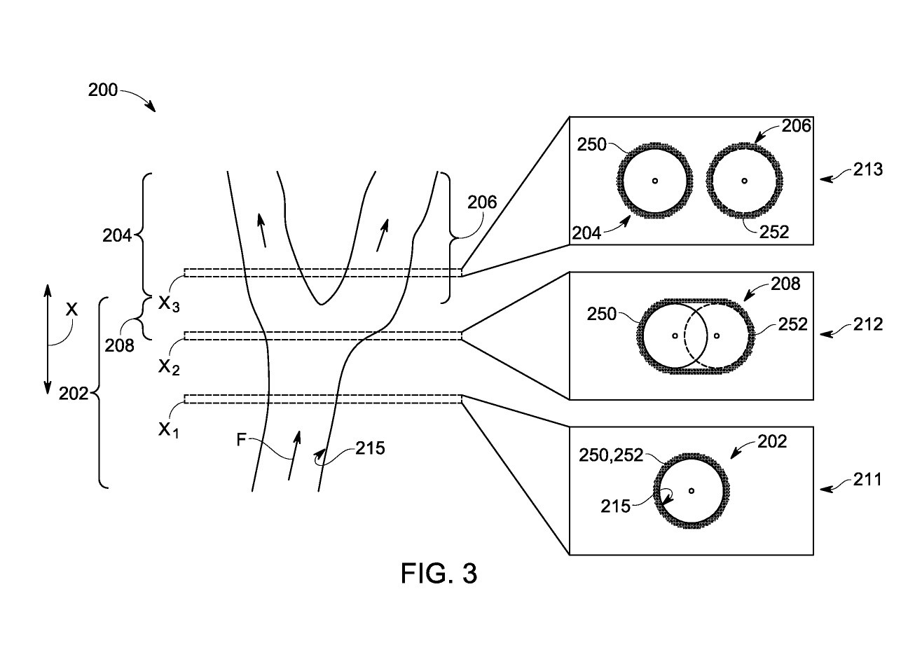

FIGS. 3-6 are described herein with reference to the method 220 shown in FIG. 2. FIG. 3 illustrates one example of a vascular structure 200 within a VOI. In certain embodiments, the vascular structure 200 is a carotid, but embodiments described herein may also be implemented with other vascular structures or other anatomical structures. As shown, the vascular structure 200 includes a main or common vessel 202, a first vessel or branch 204, and a second vessel or branch 206. The main vessel 202 may include a transition region 208 in which the main vessel 202 enlarges and begins to divide into the first and second vessels 204, 206. When the vascular structure 200 is the carotid, the main vessel 202 may be referred to as the common carotid artery (CCA), the first vessel 204 may be referred to as the internal carotid artery (ICA), and the second vessel 206 may be referred to as the external carotid artery (ECA). The transition region 208 may be referred to as the bulb region. Blood flow is indicated by arrows F and extends initially through the main vessel 202 and is then divided (within in the transition region 208) to flow through the first and second vessels 204, 206.

Although the illustrated embodiment shows only a single bifurcation in the transition region 208, it is contemplated that other embodiments may analyze vascular structures having different configurations. For example, the main vessel 202 may branch into three separate vessels. Alternatively, one of the first and second vessels 204, 206 may subsequently branch into two or more other vessels. Furthermore, although the illustrated embodiment demonstrates the blood flowing through the main vessel 202 and then being divided into the first and second vessels 204, 206, blood flow may be in the opposite direction for other vascular structures in which the blood flow from first and second vessels converges into a main vessel.

Returning to FIG. 2, the method 220 includes receiving (at 222) image data of a volume-of-interest (VOI) that includes the vascular structure. The receiving (at 222) may include receiving stored image data or may include receiving image data that is acquired in real-time, such as during an imaging session of the patient. The image data may be derived from one or more imaging modalities as described herein. In particular embodiments, the image data is ultrasound image data. The image data may include 2D, 3D, or 4D images. For example, in some embodiments, the image data includes a series of 2D images that when stacked together form a larger volume (e.g., the VOI). The 2D images may represent frames (or slices) of the VOI and may be subsequently analyzed as described herein.

In other embodiments, the image data is 3D or 4D image data. In such embodiments, the method 220 may also include analyzing the image data and apportioning or dividing the VOI into individual slices. These slices may be subsequently analyzed in a similar manner as if the slices were individual 2D images. Accordingly, the slices obtained by the imaging system (e.g., the ultrasound system) may not be the same slices that are analyzed for identifying the luminal models as set forth herein. Instead, the slices obtained by the imaging system (e.g., 2D image frames) may be combined together and re-apportioned into different slices (e.g., to obtain different thicknesses or different viewpoint of the slices) that are then analyzed as set forth herein. The slices acquired and/or analyzed may be taken substantially transverse to a flow of blood through the vascular structure.

Optionally, the method 220 includes registering (at 224) the images so that adjacent images are aligned with one another. A number of image registration algorithms may be used. For example, when the image data includes separate 2D images or frames, two or more images may be aligned by determining a transformation that minimizes a distance between the transformed target image and a reference image. The registration (at 224) may be particularly suitable for cases in which the image data is ultrasound image data. For example, adjacent 2D ultrasound images may become misaligned due to erratic movement of an ultrasound probe and/or movement of the patient. Accordingly, it may be desirable to register the 2D ultrasound images before the 2D images are analyzed to identify luminal models as set forth herein. In particular embodiments, a masked fast fourier transform (FFT) registration algorithm may be used to register selected pairs of images. The registration algorithm may be limited to translational motion.

In certain embodiments, the registration (at 224) may include registering 2D images that are a designated number of slices apart. For instance, considering that the component of highest frequency in the carotid motion is due to cardiac pulsation, the designated number of slices may be equal to 10 when the acquisition rate is 30 slices per second so that the registered images will still be within the same cardiac cycle. Translation computed in this manner may then be interpolated across the intervening slices. Such algorithms are described in greater detail in Padfield, Dirk, “Masked FFT registration,” 2010 IEEE Conference on Computer Vision and Pattern Recognition (CVPR), June 13-18, 2010, which is incorporated herein by reference in its entirety.

As described herein, embodiments may be configured to generate a vascular model based on luminal model(s) that are determined (e.g., calculated) for a plurality of slices of the VOI. Optionally, the vascular model may be generated into an image such as a surface rendering or other image of the vascular structure, which can be viewed by the user or other individual. As used herein, a “luminal model” represents at least a portion of a lumen of a designated slice (or slice-of-interest) of the VOI. In some cases, a slice will have only a single vessel and, as such, may have only a single luminal model associated with the slice. In other cases, a slice will have a plurality of vessels. Thus, the slice may have a plurality of luminal models. The luminal model(s) of each designated slice include information that corresponds to a location, shape, and size of the lumen(s) in the designated slice. Accordingly, the luminal model may include information that corresponds to (a) a location of the respective lumen in the designated slice; (b) at least a portion of a contour or shape of an interior surface that defines the lumen in the designated slice; and/or (c) at least one dimension that is indicative of a size of the corresponding lumen in the designated slice. Collectively, the information provided by the luminal model(s) may be used to generate a vascular model of the vascular structure in the VOI.

In some cases, the luminal models may include or be represented by a graphical outline that is overlaid with images of the VOI, which may be viewed by a user of the system 100 or other individual. The graphical outline may be configured to extend approximately along a wall of the vessel. For example, the wall may not be entirely defined in at least one or more medical images. Accordingly, the graphical outlines may enable a user of the system 100 to confirm that the vessel wall has been identified by the system 100.

In some embodiments, the graphical outline is a curvilinear outline that appears to be overlaid onto the image to extend approximately along the wall of the corresponding vessel. By way of example, the curvilinear outline may be a circle. In such embodiments, a location of the luminal model in the designated slice may be represented by a center point (e.g., center) of the circle in the designated slice. The center point may have coordinates (e.g., x, y coordinates) that locate the center point in the slice. For example, if the slice is 600 by 400 pixels, the value of x can be 0 to 600 and the value of y can be 0 to 400. When the graphical outline is a circle, the dimension(s) of the luminal model may include one or more of a diameter, radius, circumference, area, or other circle characteristic.

As another example, the graphical outline may be an ellipse or oval. Again, a location of the luminal model in the designated slice may be represented by a center point (e.g., center) of the ellipse in the designated slice. The dimension(s) may include one or more of a perimeter, foci, major axis length, minor axis length, or other ellipse characteristic. It should be noted that the same vessel may be represented by different types of luminal models at different slices of the VOI. For example, the same vessel may be represented by a circle in a first slice and represented by an ellipse in a subsequent slice.

In other embodiments, the graphical outline is not a curvilinear outline. For instance, the graphical outline may be at least two segments that are overlaid onto the image. The two segments may span across the lumen from one wall surface to an opposite wall surface. As a specific example, the segments may be two axes that are perpendicular to each other and intersect each other at a center of the lumen.

To illustrate the method 220, representative slices 211-213 of the VOI are shown in FIG. 3. The slice 211 includes a cross-section of the main vessel 202, the slice 212 includes a cross-section of the main vessel 202 that has the transition region 208, and the slice 213 includes cross-sections of the first and second vessels 204, 206. The slices 211-213 may be 2D medical images (e.g., 2D ultrasound images) acquired by an imaging system. Alternatively, the slices 211-213 may be generated by the system 100 after apportioning the VOI.

As shown in FIG. 3, the slices 211-213 may include graphical outlines that represent first and second luminal models 250, 252. The first and second luminal models 250, 252 may have different locations, shapes, and/or sizes. To determine the locations, shapes, and sizes of the luminal models 250, 252 in the slices 211-213, the system 100 may analyze the image data associated with the corresponding slice. For example, the method 220 may include analyzing (at 226) an initial slice of the VOI to establish initial positions of the first and second luminal models 250, 252 in the slice 211. The initialization module 132 may execute the analyzing operation (at 226) and analyze one or more initial slices of the VOI to determine an initial position (e.g., size, shape, and location) of the luminal models in the slice.

As used herein, an “initial slice” is a slice of the VOI that may be used to initiate a tracking protocol. More specifically, it is not necessary that the initial slice(s) be the very first slice or first slices in the VOI. Instead, an initial slice may be a slice that includes a readily identifiable vessel that may be used to locate the luminal models and begin the tracking protocol. For example, the initial slice 211 includes the main vessel 202. As described above, the main vessel 202 may be the CCA of the carotid. Due to the location and thickness of the CCA, the CCA typically has greater definition than the ICA and ECA and may be more readily identified. Accordingly, the CCA may be used to initialize the tracking protocol.

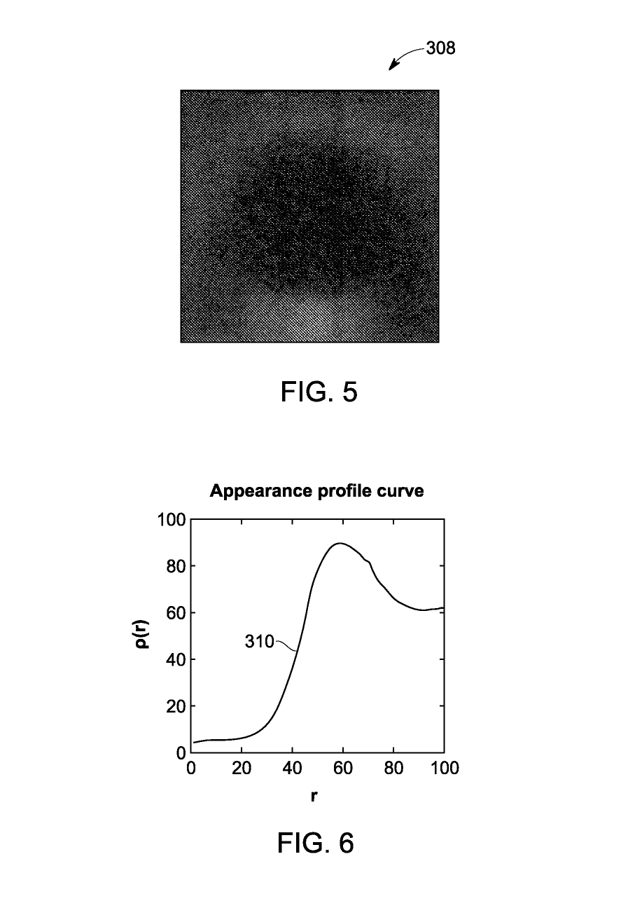

In particular embodiments, the luminal models 250, 252 in the initial slice 211 may be determined by a template-matching protocol. A template may be generated before or during implementation of the method 220. In an exemplary embodiment, the template is based on previously-acquired image data. For example, if the image data is ultrasound image data, a template may be generated by manually cropping, translating, and scaling numerous example ultrasound images that include a main vessel (e.g., the CCA). For the implementation of one embodiment, one hundred (100) ultrasound images of CCA's from other patients were manually cropped, translated, and scaled for implementing the method 220. The intensities of these images were averaged at each pixel to generate the template. An image 308 of an exemplary template is shown in FIG. 5. With the template, an initial location and dimension of the first and second luminal models 250, 252 may be identified in the initial slice 211 by matching the template against the image data of the initial slice 211. The template may be matched with more than one initial slice. In some embodiments, the shape of the template may be assumed to be a circle. In such cases, the initial location may be represented by a center point of the circle and the initial dimension may be a radius (or other circle characteristic).

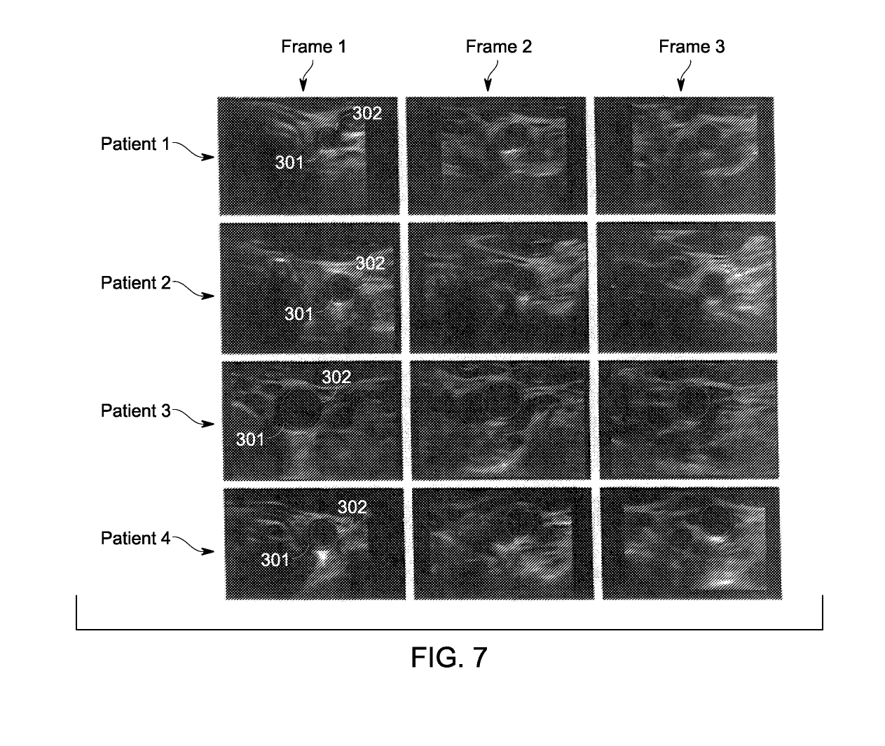

As shown in FIG. 3, the first and second luminal models 250, 252 may have the same exact locations and dimensions. In the slice 211, the first and second luminal models 250, 252 completely overlap each other such that it appears only one luminal model is shown. In other embodiments, however, the first and second luminal models 250, 252 may not overlap each other in the main vessel 202. For example, the first luminal model 250 may approximately align with a first half of the vessel wall and the second luminal model 252 may approximately align with a second opposing half of the vessel wall. Examples of this are shown in FIG. 7 in Slice 1 for Patients 1-4. As shown, each Slice 1 includes a cross-section of a CCA for the respective patient and includes non-overlapping luminal models.

Although the illustrated embodiment describes the analyzing operation (at 226) as being executed through a template-matching protocol, it is understood that other methods for identifying an initial position of the luminal models may be used in alternative embodiments.

The method 220 may also include determining (at 228) locations and dimensions of the first and second luminal models in subsequent slices. The first and second luminal models are configured to follow paths of the first and second vessels, respectively. As described below, for a designated slice of the VOI, the locations and the dimensions of the first and second luminal models of the designated slice are based on the locations and the dimensions of the first and second luminal models, respectively, in a prior slice and also the image data that corresponds to the designated slice.

In some embodiments, the first and second luminal models in subsequent slices are calculated using a model-selection function. The model-selection function may include various terms or factors to determine one or more luminal models in a slice. The model-selection function may identify a most suitable luminal model for the slice-of-interest from a finite number of potential luminal models. Terms or factors of the model-selection may include, for example: (a) a template or appearance model that represents an expectant or ideal image of the vascular structure in the slice; (b) the actual image data corresponding to the slice-of-interest; and (c) a separation term that accounts for the typical or average vascular structure.

Effectively, the model-selection function may calculate a figure of merit for each given parameter (or each set of given parameters) from a finite number of potential parameters (or sets of parameters). In particular embodiments, the model-selection function is or includes a likelihood function or a cost function. With respect to the likelihood function, a number of likelihood values or terms (hereinafter referred to as “likelihoods”) may be calculated for a finite number of potential parameters. The potential parameter with the greatest likelihood may be used to determine the luminal model(s) of the designated slice. In a similar manner, the cost function may be used to calculate a number of cost values or terms (hereinafter referred to as “costs”) for a finite number of potential parameters. The potential parameter with the least cost may be used to determine the luminal model(s) of the designated slice. In one or more embodiments, the “finite number of potential parameters” are based on the parameter(s) of a prior slice. The “prior slice” may be one or more slices and may be the slice immediately before or proximate to the slice-of-interest.

The tracking module 133 may execute the determining operation (at 228) and may be configured to calculate the locations and the dimensions of the first and second luminal models 250, 252 in the slice 215 using a model-selection function. As described herein, the model-selection function may be based, in part, on a template or appearance model. For example, the template or appearance model may be a one-dimensional radial profile. An exemplary radial profile, ρ(r) (represented as curve 310) is shown in FIG. 6 and may be used by embodiments described herein. The designated radial profile may be based on historical data. For example, the designated radial profile may be generated by averaging all of the radial profiles that emanate from a center point of an average vessel. The average vessel may be represented by, for example, the template generated by the initialization module 132 as described above and shown in FIG. 5. In other embodiments, the designated radial profile is only partially based on historical data and may be modified by a user. In some embodiments, the designated radial profile may be generated by a user based on his or her experience as to what the radial profile should be.

The designated radial profile may then be used in the model-selection function. For example, the model-selection function may determine a likelihood or cost for a given parameter (e.g., a given center point and size) for a plurality of potential parameters. The parameter that is associated with the greatest likelihood or least cost may then be used to determine the luminal model for the slice.

As a specific example, the image data corresponding to each slice includes intensity signals. More specifically, each slice may include an array of pixels in which each pixel has an intensity value. By way of example only, each slice may be about 600×400 pixels. For a given parameter θᵢ=(xᵢ, yᵢ, Rᵢ), wherein (xᵢ, yᵢ) is a center point of a corresponding lumen in the slice i and Rᵢ is a dimension (e.g., radius of a circle) of the lumen, equation (1) calculates the likelihood L(Iᵢ|θᵢ) of the image of slice i, or (Iᵢ), given the proposed center point (xᵢ, yᵢ) and the proposed dimension Rᵢ. This equation is given as:

L(Iᵢ|θᵢ)=exp(−Σ(ρᵢ(r/Rᵢ)−ρ₀(r))²).

wherein ρ₀(r) is the designated radial profile and ρᵢ(r/Rᵢ) is the actual radial profile in the slice i. The designated radial profile, as described above, may be a template or appearance model that represents an expected image of the vascular structure. This template or appearance model may be similar to or based on the template/appearance model used by the initialization module to identify the initial slice. The actual radial profile is based on the actual image data of the slice-of-interest.

Incorporating slice-to-slice smoothness constraints, a likelihood term that corresponds to a posterior slice may be obtained:

L(θᵢ|Iᵢ,θᵢ₋₁)=L(Iᵢ|θᵢ)exp(−α∥θᵢ−θᵢ₋₁∥²) (2)

wherein L(θi|Iᵢ,θᵢ₋₁) is the likelihood of the subsequent slice; α is a smoothness parameter; and ∥ƒᵢ−θᵢ₋₁∥ is a Euclidean norm.

The above equations, however, may only be appropriate for modeling a single vessel, such as the CCA. To extend the formula in order to model branching or bifurcation of vessels, the formula may be modified to track the luminal models simultaneously, but independently. For example, instead of a single parameter θᵢ, two potential parameters, θᵢ¹, θᵢ², for two vessels in a slice may be provided. The likelihood for tracking independent luminal models is the product of the individual likelihood functions. In other words, the likelihood terms for θᵢ¹, θᵢ² may be multiplied to obtain a total likelihood.

In order to avoid a degenerate solution, however, the model-selection function may be configured to account for vessel trajectories that are likely to occur based on human anatomy. For example, in the human anatomy, the ICA and the ECA typically split or project slightly away from each other at the bulb region. Accordingly, the model-selection function may include a separation factor or term. The separation term may also be described as a biasing factor or repulsive factor. The separation term predisposes the model-selection function to move the locations of the first and second luminal models away from each other as the vascular structure transitions from the main vessel to the first and second vessels. For example, this interaction may be modeled as:

φ(θᵢ¹,θᵢ²)=exp(λ∥xᵢ¹−xᵢ²∥²+∥yᵢ¹−yᵢ²∥)) (3)

wherein Φ(θᵢ¹θᵢ²) provides a repulsive interaction that forces divergence in the trajectories of the luminal models when supported by the image data, which may occur proximate to the transition region 208 (e.g., bulb region). The parameter λ controls a range of force and may be determined empirically. One example value of λ is 0.01, but others may be used. The interaction force may be stronger when the luminal models are close to each other and exponentially weaker as the luminal models become more separated.

Using the above equations, the likelihood function for tracking two luminal models within a single slice becomes:

L(θᵢ¹,θᵢ²|θᵢ₋₁¹,θᵢ₋₁²)=L(θᵢ¹|Iᵢ,θᵢ₋₁¹)L(θᵢ²|Iᵢ,θᵢ₋₁²)φ(θᵢ¹,θᵢ²) (4)

Equation (4) may be applied slice-by-slice in sequential order by the tracking module 133 by searching over a range of for θᵢ centered at θᵢ₋₁. Thus, for a subsequent slice, the tracking module 133 may use the locations of the luminal models (e.g., center points) from the prior slice and the sizes (e.g., dimensions) of the luminal models from the prior slice to identify the locations and dimensions of the first and second luminal models in the subsequent slice.

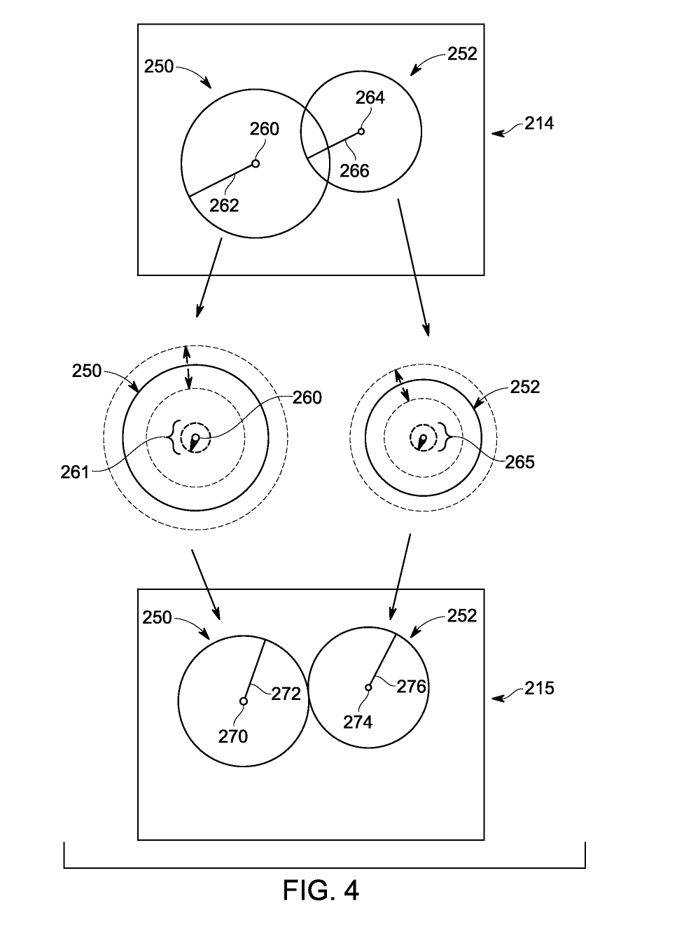

FIG. 4 visually illustrates the operation of the model-selection function applied by the tracking module 133. As shown, FIG. 4 includes a slice 214 and a subsequent slice 215. The slices 214, 215 may be located between the slices 212 and 213 shown in FIG. 3. The slice 214 may be referred to as the prior slice as the slice 214 is located before the slice 215 in the VOI. As shown, the slice 214 includes the first and second luminal models 250, 252. The first luminal model 250 has a center point 260 and a radius 262, and the second luminal model 252 has a center point 264 and a radius 266. The center points 260, 264 may be represented by coordinates (e.g., x-y coordinates) and the radii may be represented by respective values.

To determine the first and second luminal models 250, 252 in the subsequent slice 215, embodiments may calculate a plurality of likelihoods or costs that are based on potential parameters (e.g., proposed center points and radii) for each of the luminal models. The pair of potential parameters that have the greatest likelihood (or least cost) may then be used to determine the first and second luminal models in the subsequent slice 215. The potential parameters are based on the parameters of the luminal models 250, 252. More specifically, the potential parameters are limited by the actual values of the parameters in the prior slice. For example, the center point 270 of the luminal model 250 within the subsequent slice 215 may be within a predetermined area or region 261 of the center point 260 in the prior slice 214. The center point 274 of the luminal model 252 within the subsequent slice 215 may be within a predetermined area or region 265 of the center point 264 in the prior slice 214. Likewise, the radius 272 of the luminal model 250 within the subsequent slice 215 may be within a range of the radius 262 in the prior slice 214. The radius 276 of the luminal model 252 within the subsequent slice 215 may be within a range of the radius 266 in the prior slice 214. These ranges of the radii are demonstrated by the smaller and larger dashed circles shown in FIG. 4 with respect to the luminal models 250, 252. The tracking module 214 is configured to calculate the likelihoods (or costs) to determine the pair of parameters that correspond to the greatest likelihood (or least cost). The parameters associated with the greatest likelihood (or least cost) may then determine the locations and dimensions of the luminal models 250, 252 in the subsequent slice 215.

To provide a specific example, the center points 260, 264 may be (150, 150) and (290, 190), respectively. The radii 262, 266 may be 70 and 50, respectively. The tracking module 133 may be configured to calculate the likelihoods in which the values for the center points 270, 274 may be within a predefined area that is centered by the center points 260, 264. The values for the radius 272 may be within a range from 0.6(R₁) to about 1.4(R₁) in which R₁ is the value of the radius 262 or 70, and values for the radius 276 may be within a range from 0.6(R₂) to about 1.4(R₂) in which R₂ is the value of the radius 266 or 50. After applying the model-selection function to the possible parameters, it may be determined that the center points 270, 274 are, for example, (140, 200) and (300, 190), respectively. The radii 272, 276 may be, for example, 60 and 50, respectively. Accordingly, the luminal model 250 in the subsequent slice 215 may have a center point of (140, 200) and a radius of 60, and the luminal model 252 in the subsequent slice 215 may have a center point of (300, 190) and a radius of 50.

The subsequent slice 215 may then become the prior slice for determining the luminal models in another subsequent slice. In this iterative manner, the cost function may be applied slice-by-slice to track the vascular structure.

FIG. 7 illustrates image slices from four different patients (Patients 1-4) of vascular structures that were analyzed by embodiments described herein. More specifically, the carotid from four different patients was examined. FIG. 7 shows three image slices for each of the patients. Slice 1 correlates to the CCA of the carotid for each of the patients. Slice 2 correlates to the bulb region, and Slice 3 includes the ICA and ECA of the carotid for each of the patients. As shown, graphical outlines 301, 302 of the luminal models have been overlaid with the ultrasound image. Each of the slices includes two luminal models. As described above, embodiments may be used to actively acquire images of the carotid. In such cases, the system 100 may be configured to automatically generate graphical outlines of the luminal models.

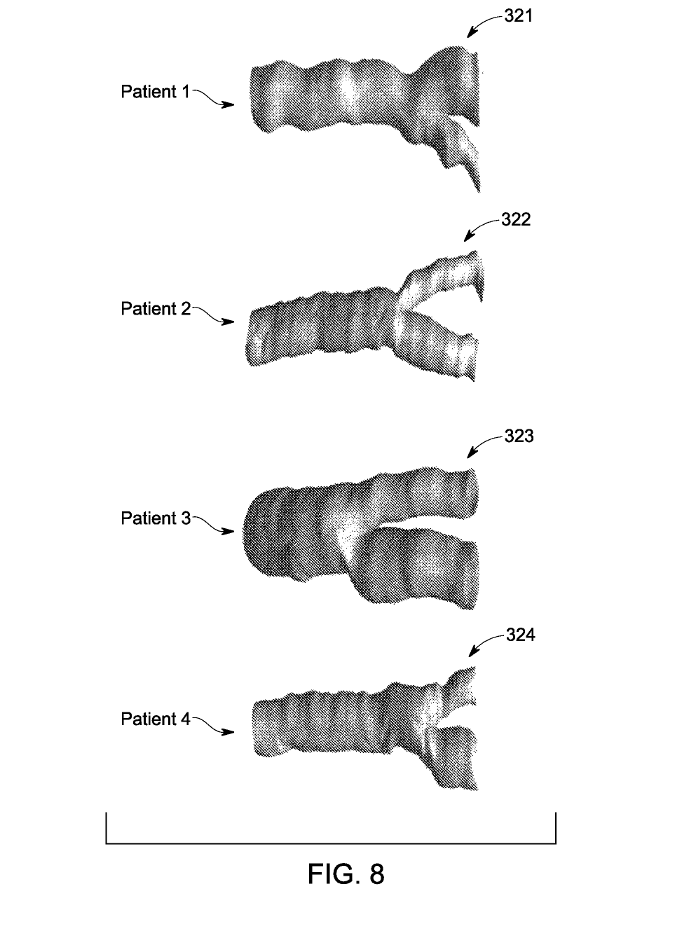

After the analyzing and determining operations (at 226 and 228), the method 220 may also include using (at 230) the locations and the dimensions of the first and second luminal models to image or analyze the vascular structure. For example, the using operation (at 230) may include at least one (a) identifying a location of a wall for at least one of the vessels; (b) generating an image of the vascular structure; (e) obtaining measurements of the vascular structure; or (d) evaluating plaque within the vascular structure. The using operation (at 230) may be executed by the analysis module 134. By way of example, FIG. 8 shows surface renderings 321-324 of the vascular structures for patients 1-4 shown in FIG. 7. The surface renderings may be generated by the system 100 and, optionally, displayed to the user of the system 100 or other individual (e.g., doctor).

As used herein, the terms “computer,” “computing system,” “system,” “system controller,” or “module” may include a hardware and/or software device or system that operates to perform one or more functions. For example, a module or system may include a computer processor, controller, or other logic-based device that performs operations based on instructions stored on a tangible and non-transitory computer readable storage medium, such as a computer memory. Some examples include microcontrollers, reduced instruction set computers (RISC), application specific integrated circuits (ASICs), and logic circuits. In some cases, a module or system may include a hard-wired device that performs operations based on hard-wired logic of the device. The modules shown in the attached figures may represent the hardware that operates based on software or hardwired instructions, the software that directs hardware to perform the operations, or a combination thereof.

Sets of instructions may include various commands that instruct the computing system or system controller as a processing machine to perform specific operations such as the methods and processes described herein. The set of instructions may be in the form of a software program or module. The software may be in various forms such as system software or application software. Further, the software may be in the form of a collection of separate programs, a program module (or module) within a larger program, or a portion of a program module. The software also may include modular programming in the form of object-oriented programming. The processing of input data by the processing machine may be in response to user commands, or in response to results of previous processing, or in response to a request made by another processing machine. The program is configured to run on both 32-bit and 64-bit operating systems. A 32-bit operating system like Windows XP™ can only use up to 3 GB bytes of memory, while a 64-bit operating system like Window's Vista™ can use as many as 16 exabytes (16 billion GB). In some embodiments, the program is configured to be executed on a Linux-based system.

As used herein, the terms “software” and “firmware” are interchangeable, and include any computer program stored in memory for execution by a computing system, including RAM memory, ROM memory, EPROM memory, EEPROM memory, and non-volatile RAM (NVRAM) memory. The above memory types are exemplary only, and are thus not limiting as to the types of memory usable for storage of a computer program.

In one embodiment, a system for analyzing a vascular structure is provided. The vascular structure has a main vessel and first and second vessels that branch from the main vessel. Each of the main vessel and the first and second vessels has a respective lumen. The system includes an initialization module configured to receive image data of a volume-of-interest (VOI) that includes the vascular structure. The VOI is represented as a series of slices that extend substantially transverse to a flow of blood through the vascular structure. The initialization module is configured to analyze a slice of the VOI that includes the main vessel to position first and second luminal models in the lumen of the main vessel. Each of the first and second luminal models represents at least a corresponding portion of a cross-sectional shape of the respective lumen and has a location in the slice and a dimension that corresponds to a size of the lumen. The system also includes a tracking module that is configured to determine the locations and the dimensions of the first and second luminal models in subsequent slices. The first and second luminal models follow paths of the first and second vessels, respectively. For a designated slice, the locations and the dimensions of the first and second luminal models of the designated slice are based on the locations and the dimensions of the first and second luminal models, respectively, in a prior slice and also the image data that corresponds to the designated slice.

In one aspect, at least one of the initialization module or the tracking module may utilize an appearance model to identify the locations and dimensions of the first and second luminal models. The appearance model may represent an expected image of the vascular structure.

In another aspect, the tracking module, for at least one of the subsequent slices, may be configured to calculate the locations and the dimensions of the first and second luminal models in the at least one subsequent slice using a model-selection function. For example, the model-selection function may be a probability distribution function.

Optionally, the tracking module may be configured to calculate likelihoods or costs for a plurality of potential parameters using the model-selection function. The potential parameters having the greatest likelihood or the least cost may be used by the tracking module to determine the first and second luminal models of the at least one subsequent slice. Also optionally, the model-selection function may include a separation term that predisposes the model-selection function to separate the locations of the first and second luminal models away from each other as the vascular structure transitions from the main vessel to the first and second vessels.

In another aspect, the locations of the first and second luminal models in the designated slice are within a predetermined region that is determined by the locations of the first and second luminal models in the prior slice.

In another aspect, each of the first and second luminal models has a center point that represents the location of the respective luminal model. The center points of the first and second luminal models in the designated slice may be within a predetermined region of the center points of the first and second luminal models, respectively, in the prior slice.

In another aspect, the system may also include an analysis module configured to at least one of (a) identify a location and a contour of a corresponding wall of at least one of the vessels; (b) generate a rendering or image of the vascular structure; (c) obtain measurements of the vascular structure; or (d) evaluate plaque within the main vessel or the first and second vessels.

In another aspect, the first and second luminal models may include curvilinear outlines that extend approximately along a corresponding wall of the corresponding vessel.

In another aspect, the first and second luminal models may include curvilinear outlines that are shaped as a circle or an ellipse or other parametric shape. The dimension may correspond to one of a diameter, radius, circumference, major axis length, or minor axis length, or other parameter of the respective parametric luminal model.

In another aspect, the image data used by the initialization and tracking modules may include ultrasound image data, wherein the initialization module may be configured to register the slices so that adjacent slices are aligned with one another. Optionally, the ultrasound image data used by the initialization and tracking modules may include a stack of two-dimensional (2D) ultrasound images.

In another embodiment, a method for analyzing a vascular structure is provided. The method includes receiving image data of a volume-of-interest (VOI) that has the vascular structure. The vascular structure has a main vessel and first and second vessels that branch from the main vessel. Each of the main vessel and the first and second vessels has a respective lumen, wherein the VOI is represented as a series of slices that are taken substantially transverse to a flow of blood through the vascular structure. The method also includes analyzing a slice of the VOI that has the main vessel to position first and second luminal models in the lumen of the main vessel. Each of the first and second luminal models represents at least a corresponding portion of a cross-sectional shape of the respective lumen and has a location in the slice and a dimension. The method also includes determining the locations and the dimensions of the first and second luminal models in subsequent slices. The first and second luminal models follow paths of the first and second vessels, respectively. For a designated slice, the locations and the dimensions of the first and second luminal models of the designated slice are based on the locations and the dimensions of the first and second luminal models, respectively, in a prior slice and also the image data that corresponds to the designated slice. The method also includes using the locations and the dimensions of the first and second luminal models to image or analyze the vascular structure.

In one aspect, determining the locations and the dimensions of the first and second luminal models in subsequent slices includes using an appearance model that represents an expected image of the vascular structure.

In another aspect, determining, for at least one of the subsequent slices, includes calculating the locations and the dimensions of the first and second luminal models in the at least one subsequent slice using a model-selection function. Optionally, the model-selection function may include a separation term that predisposes the model-selection function to separate the locations of the first and second luminal models away from each other as the vascular structure transitions from the main vessel to the first and second vessels. Also optionally, determining includes calculating likelihoods or costs for a plurality of potential parameters using the model-selection function, wherein the potential parameters having the greatest likelihood or the least cost are used to determine the first and second luminal models of the at least one subsequent slice.

In another aspect, the first and second luminal models include curvilinear outlines that extend approximately along a wall of the corresponding vessel. The method may also include displaying images of the VOI on a display as a patient is imaged and overlaying the curvilinear outlines with the images.

In another aspect, the main vessel is a common carotid artery (CCA), the first vessel is an internal carotid artery (ICA) that branches from the CCA, and the second vessel is an external carotid artery (ECA) that branches from the CCA.

In another aspect, the first and second luminal models are permitted to have the same location and the same dimension in at least one of the slices that corresponds to the main vessel.

In yet another embodiment, an ultrasound system is provided. The ultrasound system includes an ultrasound probe configured to acquire ultrasound image data of a vascular structure. The vascular structure has a main vessel and first and second vessels that branch from the main vessel. Each of the main vessel and the first and second vessels has a respective lumen. The ultrasound system may also include an initialization module configured to receive the ultrasound data of a volume-of-interest (VOI) that includes the vascular structure. The VOI is represented as a series of slices that extend substantially transverse to a flow of blood through the vascular structure. The initialization module is configured to analyze a slice of the VOI that includes the main vessel to position first and second luminal models in the lumen of the main vessel. Each of the first and second luminal models represents at least a corresponding portion of a cross-sectional shape of the respective lumen and has a location in the slice and a dimension that corresponds to a size of the lumen. The ultrasound system may also include a tracking module configured to determine the locations and the dimensions of the first and second luminal models in subsequent slices. The first and second luminal models follow paths of the first and second vessels, respectively. For a designated slice, the locations and the dimensions of the first and second luminal models of the designated slice are based on the locations and the dimensions of the first and second luminal models, respectively, in a prior slice and also the ultrasound data that corresponds to the designated slice.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein are intended to define the parameters of various embodiments, they are by no means limiting and are only example embodiments. Many other embodiments will be apparent to those of skill in the art upon reviewing the above description. The scope of the present application should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A system for analyzing a vascular structure, the vascular structure having a main vessel and first and second vessels that branch from the main vessel, each of the main vessel and the first and second vessels having a respective lumen, the system comprising:
an initialization module configured to receive image data of a volume-of-interest (VOI) that includes the vascular structure, wherein the VOI is represented as a series of slices that extend substantially transverse to a flow of blood through the vascular structure, the initialization module configured to analyze a slice of the VOI that includes the main vessel to position first and second luminal models in the lumen of the main vessel, each of the first and second luminal models representing at least a corresponding portion of a cross-sectional shape of the respective lumen and having a location in the slice and a dimension that corresponds to a size of the lumen; and
a tracking module configured to determine the locations and the dimensions of the first and second luminal models in subsequent slices, the first and second luminal models following paths of the first and second vessels, respectively, wherein, for a designated slice, the locations and the dimensions of the first and second luminal models of the designated slice are based on the locations and the dimensions of the first and second luminal models, respectively, in a prior slice and also the image data that corresponds to the designated slice.

2. The system of claim 1, wherein at least one of the initialization module or the tracking module utilizes an appearance model to identify the locations and dimensions of the first and second luminal models, the appearance model representing an expected image of the vascular structure.

3. The system of claim 1, wherein the tracking module, for at least one of the subsequent slices, is configured to calculate the locations and the dimensions of the first and second luminal models in the at least one subsequent slice using a model-selection function.

4. The system of claim 3, wherein the model-selection function is a probability distribution function.

5. The system of claim 3, wherein the tracking module is configured to calculate likelihoods or costs for a plurality of potential parameters using the model-selection function, wherein the potential parameters having the greatest likelihood or the least cost are used by the tracking module to determine the first and second luminal models of the at least one subsequent slice.

6. The system of claim 3, wherein the model-selection function includes a separation term that predisposes the model-selection function to separate the locations of the first and second luminal models away from each other as the vascular structure transitions from the main vessel to the first and second vessels.

7. The system of claim 1, wherein the locations of the first and second luminal models in the designated slice are within a predetermined region that is determined by the locations of the first and second luminal models in the prior slice.

8. The system of claim 1, wherein each of the first and second luminal models has a center point that represents the location of the respective luminal model, the center points of the first and second luminal models in the designated slice being within a predetermined region of the center points of the first and second luminal models, respectively, in the prior slice.

9. The system of claim 1, further comprising an analysis module configured to at least one of (a) identify a location and a contour of a corresponding wall of at least one of the vessels; (b) generate a rendering or image of the vascular structure; (c) obtain measurements of the vascular structure; or (d) evaluate plaque within the main vessel or the first and second vessels.

10. The system of claim 1, wherein the first and second luminal models include curvilinear outlines that extend approximately along a corresponding wall of the corresponding vessel.

11. The system of claim 10, wherein the first and second luminal models include curvilinear outlines that are shaped as a circle or an ellipse or other parametric shape, the dimension corresponding to one of a diameter, radius, circumference, major axis length, or minor axis length, or other parameters of the respective parametric luminal model.

12. The system of claim 1, wherein the image data used by the initialization and tracking modules includes ultrasound image data, wherein the initialization module is configured to registering the slices so that adjacent slices are aligned with one another.

12. The system of claim 11, wherein the ultrasound image data used by the initialization and tracking modules includes a stack of two-dimensional (2D) ultrasound images.

13. A method for analyzing a vascular structure, the method comprising:
receiving image data of a volume-of-interest (VOI) that includes the vascular structure, the vascular structure having a main vessel and first and second vessels that branch from the main vessel, each of the main vessel and the first and second vessels having a respective lumen, wherein the VOI is represented as a series of slices that are taken substantially transverse to a flow of blood through the vascular structure;
analyzing a slice of the VOI that includes the main vessel to position first and second luminal models in the lumen of the main vessel, each of the first and second luminal models representing at least a corresponding portion of a cross-sectional shape of the respective lumen and having a location in the slice and a dimension;
determining the locations and the dimensions of the first and second luminal models in subsequent slices, the first and second luminal models following paths of the first and second vessels, respectively, wherein, for a designated slice, the locations and the dimensions of the first and second luminal models of the designated slice are based on the locations and the dimensions of the first and second luminal models, respectively, in a prior slice and also the image data that corresponds to the designated slice; and
using the locations and the dimensions of the first and second luminal models to image or analyze the vascular structure.

14. The system of claim 13, wherein determining the locations and the dimensions of the first and second luminal models in subsequent slices includes using an appearance model that represents an expected image of the vascular structure.

15. The method of claim 13, wherein determining, for at least one of the subsequent slices, includes calculating the locations and the dimensions of the first and second luminal models in the at least one subsequent slice using a model-selection function.

16. The method of claim 15, wherein the model-selection function includes a separation term that predisposes the model-selection function to separate the locations of the first and second luminal models away from each other as the vascular structure transitions from the main vessel to the first and second vessels.

17. The method of claim 15, wherein determining includes calculating likelihoods or costs for a plurality of potential parameters using the model-selection function, wherein the potential parameters having the greatest likelihood or the least cost are used to determine the first and second luminal models of the at least one subsequent slice.

18. The method of claim 13, wherein the first and second luminal models include curvilinear outlines that extend approximately along a wall of the corresponding vessel, the method further comprising displaying images of the VOI on a display as a patient is imaged and overlaying the curvilinear outlines with the images.

19. The method of claim 13, wherein the main vessel is a common carotid artery (CCA), the first vessel is an internal carotid artery (ICA) that branches from the CCA, and the second vessel is an external carotid artery (ECA) that branches from the CCA.

20. The method of claim 13, wherein the first and second luminal models are permitted to have the same location and the same dimension in at least one of the slices that corresponds to the main vessel.

21. An ultrasound system comprising:
an ultrasound probe configured to acquire ultrasound image data of a vascular structure, the vascular structure having a main vessel and first and second vessels that branch from the main vessel, each of the main vessel and the first and second vessels having a respective lumen;
an initialization module configured to receive the ultrasound data of a volume-of-interest (VOI) that includes the vascular structure, wherein the VOI is represented as a series of slices that extend substantially transverse to a flow of blood through the vascular structure, the initialization module configured to analyze a slice of the VOI that includes the main vessel to position first and second luminal models in the lumen of the main vessel, each of the first and second luminal models representing at least a corresponding portion of a cross-sectional shape of the respective lumen and having a location in the slice and a dimension that corresponds to a size of the lumen; and
a tracking module configured to determine the locations and the dimensions of the first and second luminal models in subsequent slices, the first and second luminal models following paths of the first and second vessels, respectively, wherein, for a designated slice, the locations and the dimensions of the first and second luminal models of the designated slice are based on the locations and the dimensions of the first and second luminal models, respectively, in a prior slice and also the ultrasound data that corresponds to the designated slice.
